(19) United States
(12) Patent Application Publication
(10) Pub. No.: US 2024/0157335 A1
(43) Pub. Date: May 16, 2024

(54) STATIONARY PHASE FOR PURIFICATION OF CANNABIDIOL WITH HIGH-PERFORMANCE LIQUID CHROMATOGRAPHY

(71) Applicant: W.R. Grace & Co.-Conn., Columbia, MD (US)

(72) Inventors: Keling ZHANG, Pasadena, MD (US); Reno NGUYEN, Rancho Cucamonga, CA (US); Simon THOMAS, Apple Valley, CA (US)

(73) Assignee: W.R. Grace & Co.-Conn., Columbia, MD (US)

(21) Appl. No.: 18/282,491

(22) PCT Filed: Mar. 9, 2022
(86) PCT No.: PCT/US2022/019611

§ 371 Date: Sep. 15, 2023

Related U.S. Application Data
(60) Provisional application No. 63/163,735, filed on Mar. 19, 2021.

(51) Int. Cl. B01J 20/287; B01D 15/32; B01J 20/10; B01J 20/28; B01J 20/32; C07C 37/82; C07D 311/80

(52) U.S. Cl. CPC B01J 20/287; B01D 15/325; B01J 20/103; B01J 20/28004; B01J 20/28016; B01J 20/28061; B01J 20/28083; B01J 20/3204; B01J 20/3219; B01J 20/3259; B01J 20/3293; C07C 37/82; C07D 311/80; B01J 2220/52

(57) ABSTRACT

A stationary phase for high-performance liquid chromatography including monofunctional primary silane functionalized silica provides improved separation performance for the preparative or process scale purification of cannabidiol (CBD) and tetrahydro-cannabinol (THC). Silica bonded with monofunctional primary silane(s) provides more efficient separation of these molecules through higher resolution values, reduced peak broadening and lower separation impedance, thus enabling higher purity product or more efficient purification process.

### CROSS REFERENCE TO RELATED APPLICATIONS

This application claims the benefit of and priority to U.S. Provisional Application No. 63/163,735, filed on Mar. 19, 2021, the entirety of which is incorporated herein by reference.

### FIELD OF THE INVENTION

This invention relates to a stationary phase for high-performance liquid chromatography (HPLC), and more particularly, stationary phase materials containing silica bonded with silane for separation and purification of a mixture comprising cannabidiol (CBD) and tetrahydrocannabinol (THC).

### BACKGROUND

Cannabidiol (CBD), a non-psychoactive component of the hemp plant, is being increasingly promoted for its potential therapeutic benefits in pharmaceutical, nutraceutical, cosmetic, food and beverage applications. CBD, one of more than 80 compounds found in hemp, acts on the human body's endocannabinoid system, providing key health benefits such as anti-seizure, anti-anxiety, anti-inflammatory, and anti-psychotic properties. “The Biology and Potential Therapeutic Effects of Cannabidiol,” Nora D. Volkow, Director, National Institute on Drug Abuse, Presentation to Senate Caucus, Jun. 24, 2015. To achieve high purity and safe CBD products, it is critical to remove THC, a hallucinogenic and additive compound, as much as possible. CBD hemp oil must contain ≤0.3% THC for it to be legal to sell and ship across the United States.

CBD and THC are structurally similar compounds, with a small difference in polarity. Chromatographic separation techniques can be used for the separation of a CBD and THC mixture. U.S. Pat. No. 10,662,137 B2 describes a method that comprises a two-step column chromatography separation, where in the separation medium in the first step is macroporous resin, and the separation medium in the second step is polyamide resin. U.S. Pat. No. 10,301,242 B2 describes a method that comprises the use of column chromatography separation to reduce the concentration of THC, wherein the separation medium used for the column chromatography can be optionally selected from macroporous resin, MCI resin and octadecyl (C18) silane bonded silica gel.

Reverse phase chromatography is the most popular column chromatography technique. In reverse phase chromatography, the stationary phase is nonpolar and the mobile phase is polar. The typical stationary phase consists of a long-chain hydrocarbon attached to a support, while a typical mobile phase comprises mixtures of water or buffer with polar solvents such as methanol and acetonitrile. Silane bonded silica gel is one of the most popular types of stationary phase for reverse phase chromatography. Silane can vary in the hydrocarbon chain length, its structure and the number of groups that can react with the silanol groups on the surface of silica. In applications involving a corrosive mobile phase, difunctional or trifunctional silanes (silanes having two or three groups that can react with the silanol groups on the surface of silica; also referred to as “multifunctional silanes”) are preferred as they provide a more stable bonded phase against degradation. “The stability of monofunctional vs. trifunctional C18 chromatographic phases bonded to silica on exposure to aqueous trifluoroacetic acid,” William H. Leister, Lehigh University, 1992. In addition, silica bonded with multifunctional silane is also found especially suited for the separation of polycyclic aromatic hydrocarbons. “Synthesis and Characterization of polymeric C18 stationary phases for liquid chromatography,” Lane C. Sander and Stephen A. Wise, Anal. Chem., 1984, 56, 504-510.

### SUMMARY

The present invention provides stationary phase materials having improved separation performance for the preparative or process scale purification of cannabidiol (CBD). Monofunctional silanes are silanes having one group that can react with the silanol groups on the surface of silica. It has been unexpectedly discovered that silica bonded with monofunctional primary silane(s) provides more efficient separation of CBD and tetrahydrocannabinol (THC) molecules through reduced peak broadening, higher resolution values and lower separation impedance during reverse phase chromatography than multifunctional silane bonded silica materials. Thus, a stationary phase comprising a monofunctional primary silane functionalized silica enables the production of a higher purity product and a more efficient purification process in comparison with the use of multifunctional silane bonded silica which provides substantially poorer separation. Accordingly, an object of the present invention is an improved stationary phase composition to improve the effectiveness of CBD and THC separation and purification during a reverse phase chromatography process. The improved composition promotes the production of high quality and safe CBD products and improves the efficiency of a CBD production process.

Another object of the invention is an improved process for the purification of CBD using a monofunctional primary silane bonded silica as the stationary phase in reverse phase column chromatography for preparative or process scale purification of CBD and THC, and a chromatography column containing the monofunctional primary silane bonded silica as the stationary phase.

In a first aspect, the invention provides a composition for a stationary phase for purification of cannabidiol (CBD) from a separation mixture comprising cannabidiol (CBD) and tetrahydrocannabinol (THC) in reverse phase chromatography, comprising silica particles having a median particle size of about 10 to about 250 μm, a surface area of about 200 to about 600 m²/g and a pore size of about 50 to 150 Å, and a monofunctional primary silane bonded to a surface of the silica particles in an amount sufficient to provide a surface coverage of about 1 to about 3 μmoles/m² of primary silane molecules per square meter of the silica particle surface.

In some embodiments the monofunctional primary silane bonded to the surface of the silica particles is one or more monofunctional primary silanes represented by general formula (1a):

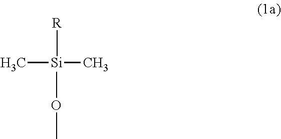

wherein R is a straight chain alkyl group with a carbon number from 6 to 30. In some embodiments R in formula (1a) is a straight chain alkyl group with a carbon number of 18.

In some embodiments, an end cap silane is bonded to the surface of the silica particles, wherein the end cap silane bonded to the surface of the silica particles is one or more end cap silane represented by formula (2) or (3), or a combination of formulas (2) and (3):

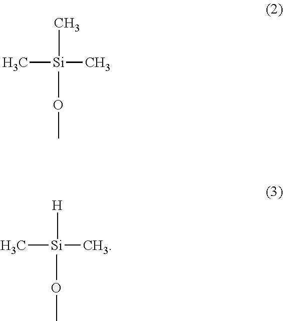

In some embodiments, the surface coverage of the monofunctional primary silane on the surface of the silica particles is about 1.5 to about 2.5 μmol/m² of primary silane molecules per square meter of the silica particle surface. In some embodiments, the total surface coverage of the monofunctional primary silane and the end cap silane on the surface of the silica particles is about 2 to about 6 μmol/m².

In another aspect, the invention provides a composition for the stationary phase for purification of a separation mixture comprising cannabidiol (CBD) and tetrahydrocannabinol (THC) in reverse phase chromatography, obtained by reacting silica particles with a monofunctional primary silane to bond the monofunctional primary silane to a surface of the silica particles, wherein the silica particles have a median particle size of about 10 to about 250 p.m, surface area of about 200 to about 600 m²/g and a pore size of about 50 to about 150 Å, wherein the monofunctional primary silane is bonded to a surface of the silica particles in an amount sufficient to provide a surface coverage of about 1 to about 3 μmoles/m² of primary silane molecules per square meter of the silica particle surface.

In some embodiments, the composition is obtained by reacting the silica particles with an end cap silane reagent after reacting the silica particles with the monofunctional primary silane, wherein the end cap silane reagent is selected from the group consisting of hexamethyl di silazane (HMDS), trimethylchlorosilane, chlorodimethylsilane, trimethylsilylimidazole, N,N-dimethylaminotrimethylsilane, and combinations thereof

In another aspect, the invention provides a method of making a stationary phase for purification of a separation mixture comprising cannabidiol (CBD) and tetrahydrocannabinol (THC) in reverse phase chromatography, comprising: reacting silica particles with a monofunctional primary silane to bond the monofunctional primary silane to a surface of the silica particles, wherein the monofunctional primary silane reacted with the silica particles is represented by general formula (1):

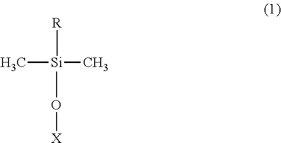

wherein R is a straight chain alkyl group with a carbon number from 6 to 30 and X is a leaving group selected from halogen, amino, methoxy or ethoxy groups, wherein silica particles have a median particle size of about 10 to about 250 μm, a surface area of about 200 to about 600 m²/g and a pore size of about 50 to about 150 Å, and wherein the monofunctional primary silane is bonded to the silica in an amount sufficient to provide a surface coverage of about 1 to about 3 μmoles/m² of primary silane molecules per square meter of silica particle surface.

In another aspect, the invention provides a method of purifying a separation mixture comprising cannabidiol (CBD) and tetrahydrocannabinol (THC) in reverse phase chromatography, comprising: supplying a mobile phase and a stream containing the separation mixture comprising CBD and THC to a chromatography column so that the mobile phase and the stream containing the separation mixture comprising CBD and THC pass through the chromatography column, and collecting CBD and THC from the chromatography column having been separated from the stream containing the separation mixture comprising CBD and THC after passing through the chromatography column, wherein the chromatography column comprises a stationary phase comprising the composition for a stationary phase of claim 1.

In some embodiments, the chromatography column has an inside diameter of about 20 mm to about 1000 mm and a length about 50 mm to about 200 cm.

In some embodiments, the separation impedance of the stationary phase for THC is about 5000 or less.

In another aspect, the invention provides a chromatography column for preparative or process scale purification of a separation mixture comprising CBD and THC, comprising the stationary phase comprising the composition for a stationary phase described above.

### BRIEF DESCRIPTION OF THE DRAWING

The FIGURE shows chromatograms of the separation of CBD and THC mixture using the stationary phase from Example 1 and Comparative Examples 1 and 2.

### DETAILED DESCRIPTION

The present invention provides high-performance functionalized stationary phase materials for use in chromatography for preparative or process scale purification of CBD. The stationary phase packing material includes silica bonded with monofunctional primary silane. The combination of desirable physical properties of the silica along with the surface functionalization with monofunctional silane provides a high-performance stationary phase. Primary silane refers to the silane that is used initially to functionalize the surface of silica gel particle. This should be distinguished from the end cap silane that is used to functionalize the remaining surface reactive silanol sites of silica surface that is not accessible to primary silane.

The inventors discovered that silica bonded with monofunctional primary silane(s) provides more efficient separation of CBD and THC through reduced peak broadening, higher resolution values and lower separation impedance, thus enabling higher purity products and a more efficient purification process. CBD and THC molecules experience significantly less peak broadening with silica bonded with monofunctional primary silane as compared to multifunctional silanes and thus they can be more effectively separated.

The stationary phase is bonded with a monofunctional primary silane. A monofunctional silane means that the silane reagent used for the functionalization reaction only has one leaving group that would react with the silanol group on the surface of silica. After the reaction, the silane is bonded to the surface of silica through forming a siloxane bond. The leaving group combines with the hydrogen of the silanol group and forms a compound that is either in the form of an off gas or liquid reaction by-product that can be removed. Since silica typically maintains a valence of four, the three other substituents are unreactive under the exposure conditions herein. The monofunctional primary silane reagent that reacts with silica particles to form the silica bonded with monofunctional primary silane can be described by the following molecule structure shown by general formula (1).

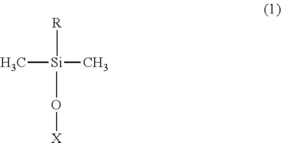

wherein R is a straight chain alkyl group with a carbon number from 6 to 30 and x is a leaving group selected from halogen, amino, methoxy or ethoxy groups. The monofunctional primary silane includes an alkyl chain, such as C6, C8, C12, C18, and C30. The monofunctional silane can be, for example, octadecyldimethylchlorosilane.

The monofunctional primary silane that is chemically bonded on the silica surface can be described by the following molecule structure shown by general formula (1a):

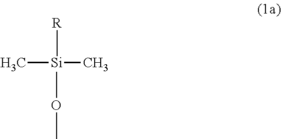

All of the monofunctional silanes bonded to the silica may be the same or combinations of different monofunctional silanes may be bonded to the silica. In an alternative embodiment, the silica is only functionalized with the monofunctional silane and optionally the end cap silane described below.

The optional end cap silane is a small molecule silane (with a low molecular mass of 90 to 170 g/mol) and is smaller than the monofunctional primary silane. The purpose of the end cap is to react with some of the remaining silanol groups on the surface of silica particle with a small molecule silane that is not accessible to the primary silane molecule. The end capped silane that is reacted onto the silica surface can be described by the following formula (2) and/or formula (3):

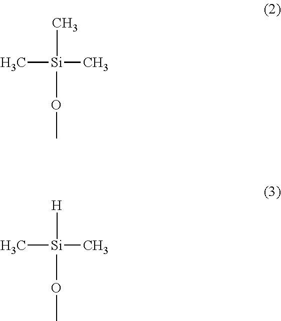

The small molecule end cap silane reagents can be, for example, hexamethyldisilazane (HMDS), trimethylchlorosilane, chlorodimethylsilane, trimethylsilylimidazole, N,N-dimethylaminotrimethylsilane, or combinations thereof

Silica gel particle, or silica gel, or silica described in this application refers to synthetic amorphous silica formed by sol gel process. It is fundamentally different from other silica material such as precipitated silica and fumed silica in the pore structure and mechanical properties Comparing to precipitated silica and fumed silica, silica gel provides well defined pore structure and high mechanical strength. Silica, Ullman's encyclopedia of industrial chemistry, 2002 Wiley-VCH Verlag GmbH & Co. KGaA.

The silica substrate used for the bonding is composed of synthetic amorphous silica gel with varying physical characteristics. Its physical characteristics including particle size, surface area and pore size have significant impact on the subsequent column chromatographic separation performance of the bonded silica, and they need to be selected carefully. The following discussion on the effect of these properties are provided in the context of reverse phase liquid chromatography.

Particle size affects the size of the interparticle channels in the packed bed of the column, which impacts mass transfer and mass diffusion, and eventually the separation efficiency N (details described in a later section). When everything else is controlled, smaller particle size provides a higher separation efficiency, N. However, particle size also affects the pressure drop across the column. Directionally, smaller particle size leads to higher pressure drop, which may result in higher energy consumption to drive the separation or even limit the use if the column housing could not withstand such high pressure. An appropriate particle size is needed to balance the need for separation efficiency and the acceptable pressure drop. For preparative and process scale purification of CBD-THC, the silica has a mean particle size (Dv50 as measured by laser scattering using Malvern instrument) ranging from about 10 to about 250 μm, preferably about 10 to about 100 μm, and most preferably about 30 to about 100

The silica gel particles are porous in structure. For porous particles, the surface area is contributed primarily by the internal surface area. Surface area affects the amount of contact and interaction between the components in the separation mixture and the stationary phase. Higher surface area is preferred to promote the interaction for better separation of the different components. However, surface area for porous particles increases with decreasing pore size. Typical surface area for silica gel particles useful in the present invention is 100 m²/g for a pore size of 300 Å and 500 m²/g for a pore size of 60Å. Diffusion of molecules slows down with smaller pore size, which can negatively impact the separation performance. For preparative and process purification of CBD-THC in the present invention, the surface area (as measured by BET method using N₂ adsorption equipment) of silica gel particles can be about 200 to about 600 m²/g, and preferably about 300 to about 600 m²/g, and most preferably about 500 to about 600 m²/g.

Pore size is important for the diffusion of analyte molecules. Pore size of the silica gel particle needs to be sufficient to allow analyte molecules to access the surface area of silica gel particles. The pore size of silica particle is also selected based on the molecular size of analytes. Smaller pore size can be used for small molecules with molecular weights of, e.g., below 1000 g/mol, and large pore size is required for large molecules. Both CBD and THC molecules have molecular weights of 314 g/mol and are considered small molecules. For preparative and process purification of CBD-THC, the pore size (as calculated from measured BET specific surface area and N₂ pore volume) of the silica particles can be about 50 to about 150Å, and preferably about 60 to about 120Å.

Other properties not discussed above include particle shape and particle size distribution. Both irregular or spherical silica can be used and no intrinsic performance advantages are identified for spherical silica particles for this use. Particle size distribution is controlled by classification techniques that are commonly used in the industry, such as sieving. Particle size distribution is inherent in the manufacturing process, except for the monodispersed particles prepared by special synthetic processes.

Details are provided about the test methods used for determining the mentioned silica properties.

### Particle Size Dv50:

Particle size of the unbonded silica gel is determined by means of laser light scattering using a Malvern 2000 with the dispersion unit Hydro 2000 G. The sample is poured into the dispersion unit filled with water free of bubbles until the obscuration reading lies in the range of 16-19%. The evaluation is conducted according to the Mie theory. Refractive index of silica gel is 1.46. Dv50, the median size for volume distribution is reported.

### BET Specific Surface Area:

BET Surface area of the unbonded silica gel is analyzed by means of N₂ adsorption using a Micromeritics TriStar 3000 instrument. Sample is first degassed at 400° C. in vacuum for 2 hours to remove any adsorbed moisture prior analysis. Surface area is determined according to Brunauer-Emmett-Teller method. (Physisorption of gases, with special reference to the evaluation of surface area and pore size distribution (IUPAC Technical Report), Pure App. Chem. 2015; 87(9-10): 1051-1069).

### N₂ Pore Volume:

N₂ Pore volume of the unbonded silica gel is analyzed using a Micromeritics Tristar 3000 instrument with N₂ gas. Sample is first degassed at 400° C. in vacuum for 2 hours to remove any adsorbed moisture prior analysis. The BJH desorption pore volume at relative pressure (P/Po) of N₂ at 0.967 is reported. The BJH desorption pore volume is the method of Barrett, Joyner, and Halenda.

### Pore Size:

Pore size referred to herein is the average pore size as determined by the following equation with the experimental determined BET specific surface area and N₂ pore volume (R. K. Iler, “The Chemistry of Silica,” Page 496, John Wiley & Sons, New York, 1979):

$$Pore size ( Å ) = \frac{4 * {N}_{2} Pore volume ( cc / g )}{BET Specific Surface area ( {m}^{2} / g )} * 1 {0}^{4}$$

Silane reacts with the surface of silica through forming covalent bonds with the silanol groups on the surface of silica. One monofunctional silane molecule only reacts with one silanol group on the surface of silica, while one multifunctional silane molecule can react with more than one silanol group when bonding onto the surface of the silica particle. Silane coverage refers to the concentration of bonded silane molecules on the surface and is determined by carbon elemental analysis and expressed in micromoles per square meter (μmol/m²). Carbon elemental analysis is conducted by a combustion method using a LECO carbon analyzer unit and is expressed in % C, meaning weight percentage of carbon in the silane bonded silica. A weighted sample is combusted at 1450° C., in an oxygen atmosphere with a vanadium oxidizing agent as accelerator. The carbon is oxidized to CO₂ and the gas is then measured by non-dispersive infrared detection. With the information of the molecular weight of silane, the number of carbon atoms in the silane molecule and molecular weight of the leaving group, the surface area of the unbonded silica gel particle, the surface coverage can be calculated accordingly and expressed in micromoles per square meter (μmol/m²).

The surface coverage of the monofunctional primary silane is about 1 to about 3 μmol/m², and this refers to about 1 to about 3 μmol of bonded primary silane molecules per square meter of silica gel surface. More preferably, the surface coverage is about 1.5 to about 2.5 μmol/m². After end capping, the total surface coverage increases to about 2 to about 6 μmol/m², and this refers to about 2 to about 6 μmol of bonded primary silane molecules and end cap silane molecules combined per square meter of silica gel surface.

The stationary phase can be formed by bonding monofunctional primary silane to silica, and optionally end capping with a small molecule silane. The bonding can be conducted with techniques that enable the reaction of the silane(s) with the silanol groups on the surface of silica. In the bonding processes, silica is first reacted with the selected primary silane and is followed by reaction with an end cap silane. However, the reaction with an end cap silane is optional.

The monofunctional silane bonded silica may be formed by, for example, an impregnation method or a solvent reflux method. Prior to bonding, silica is dried to remove physically adsorbed water. The drying temperature can be in the range of about 80° C. to about 200° C., preferably about 120° C. to about 180° C. The drying time is at least 2 hours and can be adjusted to ensure the drying step is complete and can be adjusted based on sample batch size.

In the impregnation method, a solvent, e.g., toluene, may be added to a monofunctional silane to form a silane solution. The silane solution is then added to dried silica and mixed until the liquid is absorbed into the pores of the silica. The amount of solvent can vary from zero to as much as needed for the total volume of solvent and silane to achieve 90% of the pore volume of the silica. Other solvents in addition to toluene can also be used, such as methanol, ethanol, etc. Mixing can be conducted by various equipment that are suitable for this purpose such as paddle mixer, tumbler blender or agitator. This mixing step forms a powder which is then heated while being agitated for the silane bonding reaction to occur. The heating temperature can be in the range of ambient to about 200° C., preferably about 40° C. to about 120° C., most preferably about 60° C. to about 90° C. The reaction time can be in the range of about 1 to about 48 hours, preferably about 6 to about 24 hours. Next, the small molecule end cap silane can be added to the powder and the mixture is agitated and heated for the end capping reaction to occur. The reaction time can be in the range of about 1 to about 24 hours, preferably about 1 to about 6 hours. The heating temperature can be in the range of from ambient to about 200° C., preferably about 40° C. to about 120° C., most preferably about 60° C. to about 90° C. The powder then is cooled to room temperature, washed, and dried. Washing can be conducted by a common filtration apparatus such as a Buchner funnel or other suitable equipment. The solvent used in the washing is selected to solubilize and remove any excess silane. The solvent can be optionally toluene, methanol, ethanol and other suitable solvents. Drying can be conducted by common drying equipment such as an oven or a cabinet drier with appropriate ventilation and explosion proof capabilities. Drying temperature can be in the range of ambient to about 150° C., preferably about 40° C. to about 120° C., and most preferably about 60 to about 90° C. The drying time is at least 2 hours and can be adjusted to ensure the drying step is complete and can be adjusted based on sample batch size.

In the solvent reflux method, the silica is added to a solvent such as toluene and agitated with a stirrer to form a slurry. Other solvents in addition to toluene can also be used, such as carbon tetrachloride. The slurry is heated to the reflux temperature of the solvent while continuously being stirred. The monofunctional silane is added to the slurry and the resulting slurry is refluxed for the reaction to occur and cooled prior to the next step. The reaction time can be in the range of about 1 to about 48 hours, preferably about 6 to about 24 hours. Then the small molecule end cap silane can be added and the mixture is heated to reflux for the reaction to occur and cooled prior to the next step. The reaction time can be in the range of about 1 to about 24 hours, preferably about 1 to about 6 hours. The obtained mixture is then washed and dried.

Washing can be conducted by conventional means, i.e. using a common filtration apparatus such as a Buchner funnel or other suitable equipment. The solvent used in the washing is selected to solubilize and remove any excess silane. The solvent can be optionally toluene, methanol, ethanol and other suitable solvents. Drying can be conducted by common drying equipment such as an oven or a cabinet drier with appropriate ventilation and explosion proof capabilities. Drying temperature can be in the range of ambient to about 150° C., preferably about 40° C. to about 120° C., and most preferably about 60 to about 90° C. The drying time is at least 2 hours and can be adjusted to ensure the drying step is complete and can be adjusted based on sample batch size.

The monofunctional silane bonded silica is effective for use as the stationary phase of a reverse phase chromatography column in the preparative or process scale purification of CBD from THC. The monofunctional silane bonded silica is packed into a chromatographic column. The size of the column can be any column used in preparative or process scale. For example, the column can have an inside diameter of about 20 mm to about 1000 mm and the length of the column can be, for example, from about 50 mm to about 200 cm. More specifically, for preparative scale, the inside diameter can be, for example, about 20 mm to about 100 mm, and for process scale, the inside diameter can be, for example, about 100 mm to about 1000 mm. In addition, for preparative scale, the column length can be, for example, about 100 mm to about 1000 mm and for process scale, the column length can be, for example, about 100 mm to about 2000 mm.

The mobile phase can include any organic solvent that provides suitable separation for the components in a particular process. The solvent can be, for example, methanol, ethanol, or acetonitrile. The most preferred solvents for preparatory and process scale are methanol and ethanol. The mobile phase also includes water. For example, a mixture of ethanol and water at a ratio of 65:35 (v/w) or a mixture of methanol and water at a ratio of 50:50 (v/w). The mobile phase can have a flow rate of between 5 ml/min to 100 L/min.

Monofunctional primary silane(s) bonded silica provides more efficient separation of CBD and THC, as characterized by higher resolution values between CBD and THC peaks, and lower separation impedance of THC. Higher resolution values between CBD and THC peaks provide a better separation between the two components, and lower separation impedance of THC provides a narrower THC peak at given operation pressure and analysis time to allow a more efficient separation process. For example, as seen in Table 1 below, the resolution value between CBD and THC may be about 3 or higher, and the separation impedance of THC may be about 5000 or less, preferably about 4500 or less. For purposes of the invention, the term “resolution” is used to describe the separation of two peaks and is determined as described below; the term “separation impedance” is used herein to mean the narrowness of a peak that can be achieved at a given operation pressure and analysis time and is determined as described herein below.

An explanation is provided about the parameters used to describe chromatographic separation results.

Resolution is a measure of the separation between two peaks. Higher resolution value indicates better separation of two peaks. The resolution value according to the European Pharmacopeia and Japanese Pharmacopeia method (also referred to as “EP/JP Resolution”) is determined by the following equation:

$$R = 1.18 * \vert \frac{{t}_{1} - {t}_{2}}{{W}_{1} + {W}_{2}} \vert$$

wherein R is the resolution; t₁ and t₂ are the retention time of the two peaks; and W₁ and W₂ are the peak widths at 50% peak height of the two peaks.

Column efficiency number (N), also referred as plate number, relates to a peak's retention to its width. Higher efficiency number indicates less peak broadening and better separation power of the column. Both the physical properties as well as the bonding chemistry of the stationary phase can have an impact on N. The efficiency (N) according to European Pharmacopeia and Japanese Pharmacopeia method is determined by the following equation:

$$N = 5.54 * {( \frac{t}{{W}_{50 \%}} )}^{2}$$

wherein t is the retention time of the peak, and W_{50%} is the peak width at 50% height.

To combine the measure of the narrowness of the peaks, the analysis time, and the pressure needed to drive the separation, separation impedance can also be used to describe the separation performance. (HPLC columns, theory, technology, and practice, Uwe D. Neue, 1997). Separation impedance (E) is determined by the following equation:

$$E = \frac{{t}_{0} Δ P}{{N}^{2} η}$$

wherein t₀ is the breakthrough time of an unretained peak; ΔP is the pressure drop and q is the viscosity of the mobile phase; and N is the efficiency. Higher E is desirable as it is beneficial to maximize efficiency N while minimizing analysis time and pressure drop.

### EXAMPLES

Embodiments of the invention will now be described by way of the following examples. Examples of the invention are given below by way of illustration and not by way of limitation.

### Preparation Examples

### Example 1: Preparation of Monofunctional Silane Bonded Silica using Impregnation Method

DAVISIL° FLASH 60 Å 40 μm, a commercial product, from W. R. Grace & Co. is used as the silica gel base. It has the following characteristics: Dv50 38.6 μm; BET surface area 551 m²/g and N₂ pore volume 0.9 cc/g. 9.7 ml of toluene is added to 22.5 g of octadecyldimethylchlorosilane. The obtained silane and toluene solution are added to 50 g of dried silica and mixed until the liquid is absorbed into the pores of silica. The obtained powder is heated to 80° C. while being agitated. After 24 hours, 7.5 g HMDS is added and the mixture is agitated for 24 hours at 80° C. and then cooled to room temperature. The obtained powder mixture is slurried with 150 ml of isopropyl alcohol and then filtered and washed using common filtration apparatus such as a Büchner funnel. The cake obtained after filtration is washed two times with 150 ml of isopropyl alcohol each and three times with 150 ml of methanol each. The final cake is dried in an oven at 75° C. for at least 16 hours to produce the final sample. % C analysis on the final sample is 22.6% according to carbon elemental analysis.

% C on the sample after bonding of primary silane is 19.9%, corresponding to 2.0 μmol/m² of silane coverage. % C on final sample after end capping is 22.6%, corresponding to 5.4 μmol/m² of silane coverage.

### Example 2: Preparation of Monofunctional Silane Bonded Silica using Impregnation Method

DAVISIL ° FLASH 60 Å 40 μm from W. R. Grace & Co. is used as the silica gel base. It has the following characteristics: Dv50 38.6 μm; BET surface area 551 m²/g and N₂ pore volume 0.9 cc/g. 22.5 g of octadecyldimethylchlorosilane is added to 50 g of dried silica and mixed until the silane is absorbed into the pores of silica. The obtained powder is heated to 70° C. while being agitated. After 24 hours, 7.5 g HMDS is added and the mixture is agitated for 24 hours at 70° C. and then cooled to room temperature. The obtained powder mixture is slurried with 150 ml of isopropyl alcohol and then filtered and washed using common filtration apparatus such as a Buchner funnel. The cake obtained after filtration is washed two times with 150 ml of isopropyl alcohol each and three times with 150 ml of methanol each. The final cake is dried in an oven at 75° C. for at least 16 hours to produce the final sample.

% C on the sample after bonding of primary silane is 17.9%, corresponding to 1.8 μmol/m² of silane coverage. % C on final sample after end capping is 20.0%, corresponding to 4.1 μmol/m² of silane coverage.

### Example 3: Preparation of Monofunctional Silane Bonded Silica using Solvent Reflux Method

DAVISIL® FLASH 60 Å 40 μm from W. R. Grace & Co. is used as the silica base. It has the following characteristics: Dv50 38.6 μm; BET surface area 551 m²/g and N₂ pore volume 0.9 cc/g. 50 g of dried silica is added to 0.15 liter of toluene. The mixture is agitated with a stirrer to form a slurry. The slurry is heated to reflux temperature of toluene while it is continuously being stirred. 22.5 g of octadecyldimethylchlorosilane is added to the slurry. The resulting slurry is refluxed for 9.5 hours then allowed to cool to room temperature. 7.5 g of HMDS is added and the mixture is heated to reflux for 2 hours, then cooled to room temperature. The obtained mixture is filtered and washed using common filtration apparatus such as a Buchner funnel. The cake obtained after filtration is washed two times with 150 ml of isopropyl alcohol each and three times with 150 ml of methanol each. The final cake is dried in an oven at 75° C. for at least 16 hours to produce the final sample. % C analysis on the final sample is 13.6% according to carbon elemental analysis.

### Comparative Example 1: Preparation of Multifunctional Silane Bonded Silica using Impregnation Method

DAVISIL ® FLASH 60 Å 40 μm from W. R. Grace & Co. is used as the silica gel base. It has the following characteristics: Dv50 38.6 μm; BET surface area 551 m²/g and N₂ pore volume 0.9 cc/g. 12.3 ml of toluene is added to 22.5 g of octadecyltrichlorosilane. The obtained silane and toluene solution are added to 50 g of dried silica and mixed until the liquid is absorbed into the pores of silica. The obtained powder is heated to 80° C. while being agitated. After 24 hours, 7.5 g HMDS is added and the mixture is agitated for 24 hours at 80° C. and then cooled to room temperature. The obtained powder mixture is slurried with 150 ml of isopropyl alcohol and then filtered and washed using common filtration apparatus such as a Buchner funnel. The cake obtained after filtration is washed two times with 150 ml of isopropyl alcohol each and three times with 150 ml of methanol each. The final cake is dried in an oven at 75° C. for at least 16 hours to produce the final sample. % C analysis on the final sample is 20.0% according to carbon elemental analysis.

### Comparative Example 2: Preparation of Trifunctional Silane Bonded Silica using Solvent Reflux Method

This material is functionalized with octadecyltrichlorosilane and end capped with HMDS using a solvent reflux method similar to the solvent reflux method described in Example 3. The silica base has the following characteristics: Dv50 52.6 μm; BET surface area 535 m²/g and N₂ pore volume 0.8 cc/g. % C on the final functionalized product is 17.2%.

### Comparative Example 3: Preparation of Trifunctional Silane Bonded Silica using Solvent Reflux Method

This material is functionalized with octadecyltrichlorosilane and end capped with HMDS using a solvent reflux method similar to the solvent reflux method described in Example 3. The silica base has the following characteristics: Dv50 40.8 μm; BET surface area 541 m²/g and N₂ pore volume 0.9 cc/g. % C on the final functionalized product is 16.5%.

### Comparative Example 4: Preparation of Trifunctional Silane Bonded Silica using Solvent Reflux Method

This material is functionalized with octadecyltrichlorosilane and end capped with HMDS using a solvent reflux method similar to the solvent reflux method described in Example 3. The silica base has the following characteristics: Dv50 33.0 μm; BET surface area 330 m²/g and N₂ pore volume 1.0 cc/g. % C on the final functionalized product is 15.5%.

### Testing Results

### (I) Separation of Mixture of CBD and THC

The samples from Examples 1 to 3 and Comparative Examples 1 to 4 are separately packed into chromatographic columns and tested for their effectiveness in separating CBD and THC from a mixture. The columns have an I.D. of 4.6 mm and length of 150 mm. The obtained columns are tested with the following conditions:
Mobile phase: 65:35 ethanol/water (v/w)
Sample injection volume (μL): 10
UV wavelength (nm): 220
Mobile Phase flow rate (ml/min): 1.0

UV wavelength is selected accordingly based on the UV light absorption behavior of the compounds to be analyzed, in order to best quantify their concentrations. The CBD-THC mixture contains 0.33 mg/ml of each of CBD and THC in 65:35 ethanol/water (v/w). Viscosity of 65:35 ethanol/water (v/w) at 20° C. is 2.7 cps. The separation performance of CBD-THC mixture with the different functionalized silicas from the Examples 1 to 3 and Comparative Examples 1 to 4 is summarized in Table 1. Comparing the samples of Examples 1 to 3 (bonded with C18 monofunctional silane) with the samples of the Comparative Examples 1 to 4 (bonded with C18 trifunctional silane), the samples of Examples 1 to 3 provide a higher resolution value for CBD and THC, indicating a better separation between CBD and THC components. In the specific test conditions for comparison purpose only, samples of Examples 1 to 3 provide a resolution value consistently at or above 3, which is higher than those can be achieved by comparative samples. The resolution value for CBD and THC peaks of the samples of Examples 1 to 3 are consistently above 3.0, and higher than that of all comparative samples, indicating more superior separation of the two components. In addition, comparing the samples of Examples 1 to 3 (bonded with C18 monofunctional silane) with the samples of the Comparative Examples 1 to 4 (bonded with C18 trifunctional silane), the samples of Examples 1 to 3 provide a lower separation impedance for both THC and CBD. Lower separation impedance is desirable in separation process, as it indicates narrower peaks can be achieved at a given separation pressure and analysis time.

TABLE 1
Chromatographic separation of CBD-THC with trifunctional and monofunctional silane bonded silica stationary phases

| | | | | | | | EP/JP | | | | | |
|---|---|---|---|---|---|---|---|---|---|---|---|---|
| | | | Retention | | Peak Symmetry | | Resolution | EP/JP Efficiency | | Pressure | Separation | Separation |
| | Primary | Final | Time (min.) | | (10% Height) | | CBD/ | (N/m) | | Drop | Impedance, | Impedance, |
| Sample | Silane | % C | CBD | THC | CBD | THC | THC | CBD | THC | (psi) | E, THC | E, CBD |
| Example 1 | monofunctional | 22.6 | 10.78 | 25.14 | 1.07 | 1.06 | 3.46 | 1733 | 2120 | 73 | 3235 | 4841 |
| Example 2 | silane | 20.0 | 9.63 | 22.23 | 1.06 | 1.07 | 3.46 | 1773 | 2167 | 75 | 3181 | 4752 |
| Example 3 | | 13.6 | 10.42 | 21.45 | 1.07 | 1.06 | 3.02 | 1807 | 2127 | 81 | 3566 | 4941 |
| Comparative | multifunctional | 20.0 | 8.38 | 18.11 | 1.09 | 1.07 | 2.77 | 1333 | 1607 | 71 | 5476 | 7959 |
| Example 1 | silane | | | | | | | | | | | |
| Comparative | | 17.2 | 9.37 | 21.06 | 1.16 | 1.07 | 1.98 | 582 | 773 | 52 | 17334 | 30578 |
| Example 2 | | | | | | | | | | | | |
| Comparative | | 16.5 | 11.27 | 24.75 | 1.10 | 1.10 | 2.77 | 1213 | 1593 | 65 | 5102 | 8799 |
| Example 3 | | | | | | | | | | | | |
| Comparative | | 15.5 | 4.80 | 9.23 | 1.04 | 1.04 | 2.27 | 1307 | 1413 | 64 | 6385 | 7462 |
| Example 4 | | | | | | | | | | | | |

t₀ (breakthrough time of an unretained peak) is needed in order to determine separation impedance. t₀ of this column is 78 seconds and is determined by chromatographic test and according to the following test conditions:
Mobile phase: 60:40 acetonitrile/water (v/w)
Sample injection volume (μL): 10
UV wavelength (nm): 254
Mobile phase flow rate: 1 ml/min
Test Compounds: 0.02 mg/ml uracil in mixture of methanol and water (3:1, v/v).

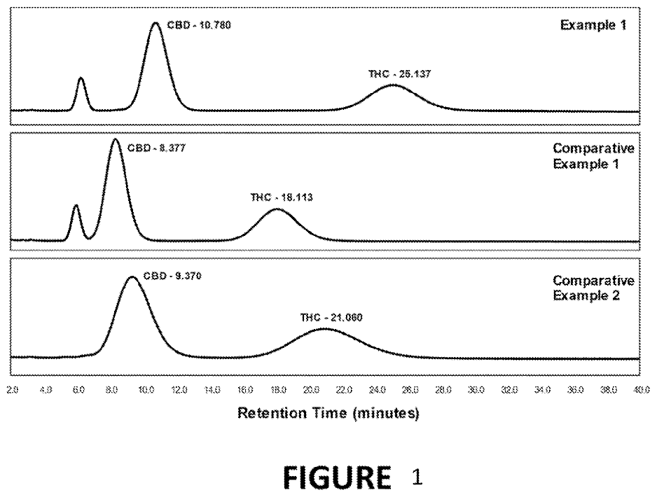

The chromatograms of the separation of CBD and THC mixture using stationary phase from Example 1 and Comparative Examples 1 and 2 are shown in FIG. 1. In comparison to Comparative Examples 1 and 2, Example 1 displays the maximum separation between the CBD and THC peaks.

While certain embodiments have been described, these embodiments have been presented by way of example only and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1-29. (canceled)

30. A method of purifying a separation mixture comprising cannabidiol (CBD) and tetrahydrocannabinol (THC) in reverse phase chromatography, comprising:
supplying a mobile phase and a stream containing the separation mixture comprising CBD and THC to a chromatography column so that the mobile phase and the stream containing the separation mixture comprising CBD and THC pass through the chromatography column; and
collecting CBD and THC from the chromatography column having been separated from the stream containing the separation mixture comprising CBD and THC after passing through the chromatography column;
wherein the chromatography column comprises a stationary phase comprising a composition comprising silica particles having a median particle size of about 10 μm to about 250 μm, a surface area of about 200 m²/g to about 600 m²/g, and a pore size of about 50 to 150 Å; and a monofunctional primary silane bonded to a surface of the silica particles in an amount sufficient to provide a surface coverage of about 1 μmoles/m² to about 3 μmoles/m² of primary silane molecules per square meter of the silica particle surface.

31. The method of purifying the separation mixture of claim 30, wherein the monofunctional primary silane bonded to the surface of the silica particles is one or more monofunctional primary silanes represented by general formula (1a): 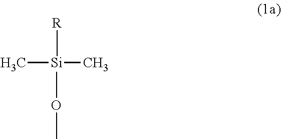
wherein R is a straight chain alkyl group with a carbon number from 6 to 30.

32. The method of purifying the separation mixture of claim 30, wherein the silica particles have a particle size Dv50 of about 30 to about 60 μm, surface area of about 400 to about 600 m²/g, and a pore size of about 60 to about 120 Å.

33. The method of purifying the separation mixture of claim 31, wherein R in formula (1a) is a straight chain alkyl group with a carbon number of 18.

34. The method of purifying the separation mixture of claim 30, further comprising an end cap silane bonded to the surface of the silica particles,
wherein the end cap silane bonded to the surface of the silica particles is one or more end cap silane represented by formula (2) or (3), or a combination of formulas (2) and (3):

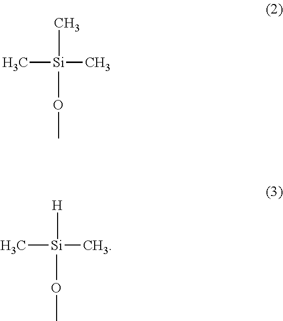

35. The method of purifying the separation mixture of claim 30, wherein the surface coverage of the monofunctional primary silane on the surface of the silica particles is about 1.5 to about 2.5 μmol/m² of primary silane molecules per square meter of the silica particle surface.

36. The method of purifying the separation mixture of claim 34, wherein the total surface coverage of the monofunctional primary silane and the end cap silane on the surface of the silica particles is about 2 to about 6 μmol/m².

37. The method of purifying the separation mixture of claim 30, wherein the chromatography column has an inside diameter of about 20 mm to about 1000 mm and a length about 50 mm to about 200 cm.

38. The method of purifying the separation mixture of claim 30, wherein the separation impedance of the stationary phase for THC is about 5000 or less.

39. A composition comprising:
silica particles having a median particle size of about 10 to about 250 μm, a surface area of about 200 to about 600 m²/g, and a pore size of about 50 to 150 Å; and
a monofunctional primary silane bonded to a surface of the silica particles in an amount sufficient to provide a surface coverage of about 1 to about 3 μmoles/m² of primary silane molecules per square meter of the silica particle surface;
wherein the composition is for use in a stationary phase for purification of cannabidiol from a separation mixture comprising cannabidiol and tetrahydrocannabinol in reverse phase chromatography.

40. A method of making a stationary phase for purification of a separation mixture comprising cannabidiol and tetrahydrocannabinol in reverse phase chromatography, comprising:
reacting silica particles with a monofunctional primary silane to bond the monofunctional primary silane to a surface of the silica particles;
wherein:
the monofunctional primary silane reacted with the silica particles is represented by general formula (1): 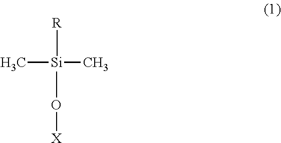
R is a straight chain alkyl group with a carbon number from 6 to 30 and X is a leaving group selected from halogen, amino, methoxy, or ethoxy groups;
silica particles have a median particle size of about 10 to about 250 μm, a surface area of about 200 to about 600 m²/g, and a pore size of about 50 to about 150 Å; and
the monofunctional primary silane is bonded to the silica in an amount sufficient to provide a surface coverage of about 1 to about 3 μmoles/m² of primary silane molecules per square meter of silica particle surface.

41. The method of claim 40, wherein the silica particles have a median particle size of about 30 to about 60 μm, a surface area of about 400 to about 600 m²/g, and a pore size of about 60 to about 120 Å.

42. The method of claim 40, wherein R in formula (1) is a straight chain alkyl group with a carbon number of 18.

43. The method of claim 40, further comprising reacting the silica particles with an end cap silane reagent after reacting the silica particles with the monofunctional primary silane, wherein the end cap silane reagent is selected from the group consisting of hexamethyldisilazane (HMDS), trimethylchlorosilane, chlorodimethylsilane, trimethylsilylimidazole, N,N-dimethylaminotrimethylsilane, and combinations thereof
